(19) United States
(12) Patent Application Publication
(10) Pub. No.: US 2008/0153120 A1
(43) Pub. Date: Jun. 26, 2008

(54) Salt Taste Receptor and its Use in an Assay for Salt Taste

(75) Inventors: Johannes LeCoutre, Pully (CH); Christopher Marc Parry, Surry (GB)

Correspondence Address: BELL, BOYD & LLOYD LLP, P.O. Box 1135, CHICAGO, IL, 60690, US

(73) Assignee: NESTEC S.A., Vevey (CH)

(21) Appl. No.: 11/815,596

(22) PCT Filed: Feb. 7, 2006
(86) PCT No.: PCT/EP06/01075

§ 371 Date: Aug. 6, 2007

(30) Foreign Application Priority Data
Feb. 7, 2005 (US) 60650940

(51) Int. Cl. C12Q 1/37; C12N 5/00; C12N 1/00; C12Q 1/02

(52) U.S. Cl. 435/23; 435/325; 435/317.1; 435/29

(57) ABSTRACT

A functional human salt taste receptor and a cell based assay that simulates human salt taste stimulation is disclosed. A method for the identification of enhancers or modulators of salt taste and food products that contain them is also disclosed. The method for the production of food products with desirable flavor properties having greatly reduced salt concentrations are disclosed. Such foods can provide substantial health benefits in many circumstances thereby providing substantial health benefit.

### BACKGROUND OF THE INVENTION

Salt taste is thought to be mediated, in part, by the Epithelial Sodium Channel (ENaC). The classical ENaC sodium channel model system is derived from kidney cells and consists of three protein subunits, ENaC-α, ENaC-β and ENaC-γ. It is thought that the functional kidney ENaC ion channel exists as an α₂βγ hetero-tetramer. A fourth protein subunit, ENaC-γ, has been identified but its function remains unknown.

Mouse kidney derived ENaC sensitivity is increased by certain channel activating proteases (mCAP1, mCAP2 and mCAP3). These proteases are expressed in the same tissues as ENaC, including kidney, lung, colon, small intestine and stomach tissues and are thought to activate the ion channel by increasing the amount of time the channel is in an open conformation.

Kidney ENaC is inhibited by the diuretic amiloride (N-amidino-3,5-diamino-6-chloropyrazine carboxamide). Amiloride would be expected to interfere with salt taste if ENaC is the dominant ion channel involved in salt taste and, in fact, the amiloride effect is clearly observed in rodents. However, the effect is only seen in some humans suggesting the existence of different receptor, or receptor configuration.

Thus, there remains a need in the art for the identification and preparation of sodium channels that are involved in human salt taste. Such a system could be used to identify compounds that either enhance or inhibit the perception of salt taste.

### SUMMARY OF THE INVENTION

The present invention provides a functional human salt taste receptor and a cell based assay that simulates human salt taste stimulation. The invention further provides for the identification of enhancers or modulators of salt taste and food products that contain them. The invention also provides for the production of food products that retain desirable flavor properties although they contain greatly reduced salt concentrations. Such foods can provide substantial health benefits in many circumstances.

In an embodiment, the present invention includes an assay that simulates human salt taste stimulation. Methods for detecting sodium ion flux in cells are known and can be utilized to determine sodium flux in the presence of various unknown compounds in order to identify which of those compounds influence salt taste perception.

In an embodiment, the invention provides an assay that simulates human salt taste stimulation that utilizes cells that express a functional sodium ion channel.

In an embodiment, the invention provides an assay that simulates human salt taste stimulation utilizing cells that express a functional sodium ion channel from a recombinant DNA molecule.

The invention also provides a method for identifying modulators of salt taste, incubating the cell with a compound and determining sodium ion flux through the sodium ion channel in the cell.

The invention further provides a method for preparing a food product by identifying modulators of salt taste as set forth above and identifying those modulators that increase sodium ion flux through the sodium ion channel of the cell and adding the compound to a food product.

In an embodiment, the invention provides a recombinant DNA molecule that includes the genes for ENaC-α, ENaC-β, ENaC-γ or ENaC-δ and further includes a gene for either hCAP1 or hCAP3.

Additional features and advantages of the present invention are described in, and will be apparent from, the following Detailed Description of the Invention.

### BRIEF DESCRIPTION OF FIGURES

FIG. 1 provides a schematic representation of CAP acting on ENaC increasing its open conformation which provides enhanced sodium flux. H, D and S represent the amino acids in the protease active site.

FIG. 2 is a 1.2% agarose gel of the PCR amplification products of human non-taste tissue cDNA library (NT) and a taste cell cDNA library (T) using hCAP1-3 (SEQ ID Nos. 1-6) and vector control primers. Lanes 2 to 7 have as template for the PCR reaction from left to right: water, non-taste tissue library DNA and taste cell library DNA. Lane 1: left −1 μg 100 bp ladder (Invitrogen), right μg 1 Kb ladder (Invitrogen), lane 2: hCAP1 (SEQ ID No. 3) and T7 vector primer, lane 3: hCAP2 (SEQ ID No. 3) and T7 vector primer, lane 4: hCAP3 (SEQ ID No. 5) and T7 vector primer, lane 5: hCAP1 (SEQ ID No. 2) and SP6 vector primer, lane 6: hCAP2 (SEQ ID No. 4) and SP6 vector primer, lane 7: primers hCAP3 (SEQ ID No. 6) and SP6 vector primer.

### DETAILED DESCRIPTION OF THE INVENTION

The present invention is based on the discovery of human equivalents of mCAP1 and mCAP3 in a human taste cell library indicating that the expression of these proteases is involved in human salt taste perception mediated by ENaC. Co-expression of these proteases with the ENaC subunits allows physiologically correct maturation and processing of the receptor complex and provides for the correct function of ENaC in cell based assays.

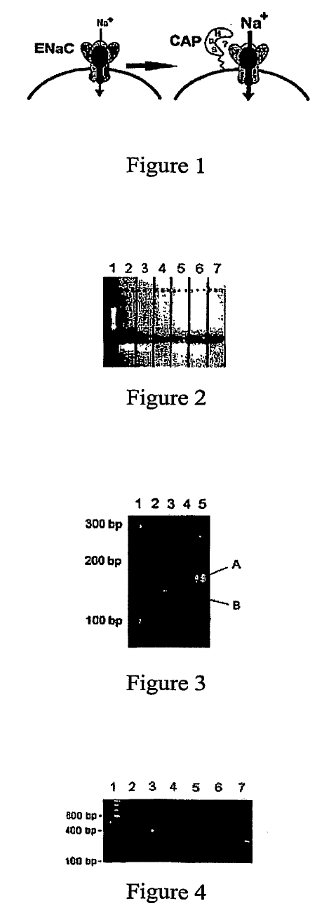

The sequences of cDNA for human equivalents of mCAP1, mCAP2 and mCAP3, as described by Vuagniaux et al. 2002 J. Gen. Physiol. (See FIG. 1B of Vuagniaux et al.), were obtained from sequence databases. The sequences are described below:

The sequences are as follows:
The human equivalent of mouse CAP1 is termed PROSTASIN or Homo sapiens protease, serine 8 (PRSS8), (accession number NM_{—}002773),
The human equivalent of mouse CAP2 is termed TMPRSS4, TMPRSS3 or MTSP2 of which there are 2 transcript variants (variant 1 accession number NM_{—}019894, variant 2 accession number NM_{—}183247). Variant 2 uses an alternate in-frame splice site in the 5′-coding region and lacks an exon in the 3′-coding region, compared to variant 1. The resulting protein (isoform 2) is shorter and has distinct N— and C-termini, compared to isoform 1,
The human equivalent of mouse CAP3 is termed MT-SP1, HAI, MTSP1, SNC19, MTSP1, TADG-15 or PRSS14 (accession number NM_{—}021978).

For purposes of this application the human equivalents of mCAP1-3 are termed hCAP1-3, respectively.

Oligonucleotide PCR primer pairs that anneal to regions corresponding to the extreme end of the 3′-untranslated region were designed such that they would be able to amplify a product from genomic DNA as well as cDNA. The oligonucleotide primer pairs are shown below in Table 1.

TABLE 1

| | | |
|---|---|---|
| Gene | SEQ ID NO. | Sequence |

| | | |
|---|---|---|
| hCAP1 F | 1 | CCCATCTTGATCTTTGAGCC |
| hCAP1 R | 2 | ATTTCTGCCCTGTTACTCCC |
| hCAP2 F | 3 | ACAGCCTCAGCATTTCTTGG |
| hCAP2 R | 4 | GCTCTTTAATAATAGTGGCC |
| hCAP3 F | 5 | AATCTCCAGGGCTCCAAATC |
| hCAP3 R | 6 | TACACACACTGAAGTCCACC |

PCR conditions were optimized by standard methods using human genomic DNA as a template. Primer pairs for hCAP1-3 all amplified a product of the expected size when genomic DNA was used as a template.

A human taste cell library has been described in Ilegems M. et al. (submitted). The optimized conditions were used to probe a human taste cell cDNA library using the 3′-gene specific primers with both T7 or SP6 vector primers in order to amplify the largest fragments contained within the library. Products of PCR reactions using the taste cell libraries were separated by agarose gel electrophoresis.

PCR amplifications of a human taste cell cDNA library were carried out for human CAP protease. Products of the expected size were obtained for hCAP1 and hCAP3. These PCR products were extracted from the gel, cloned in to pGEM-Teasy and sequenced. The sequences obtained matched those of the respective hCAP cDNAs and indicated that hCAP1 and hCAP3 are expressed in human taste cells. In addition to identifying and preparing a novel ENaC configuration, the activity of the channel activating proteases can be used to produce a correctly functioning salt taste receptor.

In view of the above, a recombinant DNA expression cassette containing hCAP1 and hCAP3 and/or ENaC-α, ENaC-β, ENaC-γ and ENaC-δ can be created using standard methods and can be expressed in various cells including eukaryotic cells by standard methods. The novel cells expressing human ENaC sodium channels and the CAP proteases can be used in known cellular assays for salt taste perception. A heterologous expression system using the eukaryotic cells would be designed to express ENaC and CAP proteases. The proteases ensure that ENaC is processed into it's taste relevant configuration. Alternatively, normal ENaC expressing cells can be treated externally with proteases to achieve this processing. Once ready, the cells will be used to measure sodium influx by known methods in the presence of compounds to be tested for their sodium influx potential which corresponds to salt taste enhancing potential. The assay, in turn, will provide for the identification of compounds that either enhance or inhibit the taste of salt. Such compounds can be included in food products in order to maintain suitable flavor over widely varying salt concentrations.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A functional sodium ion channel in a cell containing a recombinant DNA molecule, the recombinant DNA molecule comprises a gene selected from the group of genes consisting of hCAP1, hCAP3, ENaC-α, ENaC-β, ENaC-γ and ENaC-δ.

2. The functional sodium ion channel of claim 1, wherein the recombinant DNA molecule comprises at least two genes selected from the group of genes consisting of hCAP1, hCAP3, ENaC-α, ENaC-β, ENaC-γ and ENaC-δ.

3. The functional sodium ion channel of claim 1, wherein the recombinant DNA molecule comprises at least three genes selected from the group of genes consisting of hCAP1, hCAP3, ENaC-α, ENaC-β, ENaC-γ and ENaC-δ.

4. The functional sodium ion channel of claim 1, wherein the recombinant DNA molecule comprises at least four genes selected from the group of genes consisting of hCAP1, hCAP3, ENaC-α, ENaC-β, ENaC-γ and ENaC-δ.

5. The functional sodium ion channel of claim 1, wherein the recombinant DNA molecule comprises at least five genes selected from the group of genes consisting of hCAP1, hCAP3, ENaC-α, ENaC-β, ENaC-γ and ENaC-δ.

6. The functional sodium ion channel of claim 1, wherein the recombinant DNA molecule comprises hCAP1, hCAP3, ENaC-α, ENaC-β, ENaC-γ and ENaC-δ.

7. A cell comprising a recombinant DNA molecule comprising at least two genes selected from the group of genes consisting of hCAP1, hCAP3, ENaC-α, ENaC-β, ENaC-γ and ENaC-δ.

8. The cell of claim 7 wherein the cell is a eukaryotic cell.

9. An assay that simulates human salt taste stimulation comprising incubating a cell that expresses hCAP3 and produces a functional sodium ion channel, with a compound and determining ion flux in the cell.

10. The assay of claim 9, wherein the cell comprises a functional sodium ion channel comprising an expressed gene selected from the group consisting of hCAP1 and hCAP3.

11. The assay of claim 9, wherein the gene is expressed on a recombinant DNA molecule.

12. The assay of claim 9, wherein the cells are treated with proteases and utilized in the salt taste assay.

13. A method for identifying modulators of salt taste comprising:
obtaining a cell that expresses a gene selected from the group consisting of hCAP1 and hCAP3 from a recombinant DNA molecule and that produces a functional sodium ion channel,
incubating the cell with a compound, and
determining sodium ion flux in the cell.

14. A method for preparing a food product comprising:
obtaining a cell that expresses a gene selected from the group consisting of hCAP1 and hCAP3 from a recombinant DNA molecule and produces a functional sodium ion channel,
incubating the cell with an edible compound and determining whether the compound modulates sodium ion flux in the cell, and
adding the compound to a food product.

15. A method for human salt taste stimulation comprising the steps of incubating a cell that expresses hCAP3 and produces a functional sodium ion channel, with a compound and determining ion flux in the cell.


SEQUENCE LISTING (6 sequences)

SEQ ID NO: 1 - DNA, 20 bp, Homo sapiens
cccatcttgatctttgagcc

SEQ ID NO: 2 - DNA, 20 bp, Homo sapiens
atttctgccctgttactccc

SEQ ID NO: 3 - DNA, 20 bp, Homo sapiens
acagcctcagcatttcttgg

SEQ ID NO: 4 - DNA, 20 bp, Homo sapiens
gctctttaataatagtggcc

SEQ ID NO: 5 - DNA, 20 bp, Homo sapiens
aatctccagggctccaaatc

SEQ ID NO: 6 - DNA, 20 bp, Homo sapiens
tacacacactgaagtccacc